(19) United States
(12) Patent Application Publication
(10) Pub. No.: US 2007/0060539 A1
(43) Pub. Date: Mar. 15, 2007

(54) Shc proteins as therapeutic targets in proliferative diseases

(75) Inventors: A. Raymond Frackelton JR., Rumford, RI (US); Pamela A. Davol, Swansea, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Roger Williams Hospital, Providence, RI (US)

(21) Appl. No.: 11/511,073

(22) Filed: Aug. 28, 2006

Related U.S. Application Data
(63) Continuation of application No. 10/687,396, filed on Oct. 15, 2003, now abandoned.
Application No. 10/687,396 is a continuation-in-part of application No. 10/376,538, filed on Feb. 28, 2003.
(60) Provisional application No. 60/360,758, filed on Mar. 1, 2002.

(51) Int. Cl. A61K 48/00; C12Q 1/68; G01N 33/574

(52) U.S. Cl. 514/44; 435/6; 435/7.23

(57) ABSTRACT

This invention provides methods for treating a subject afflicted with a proliferative disorder comprising administering to the subject a therapeutically effective amount of an agent which inhibits the expression of p46 Shc and/or p52 Shc, inhibits the activity of p46 Shc and/or p52 Shc in the subject, or increases the level of phosphorylated p66 Shc in the subject. This invention also provides methods for determining whether an agent inhibits the phosphorylation of p46 Shc or p52 Shc; inhibits the dephosphorylation of p66 Shc comprising; or an agent inhibits the binding of a Shc A protein with a protein to which the Shc A protein must bind in a cell in order to carry out its proliferative function. This invention also provides articles of manufacture for use in treating a proliferative disorder in a subject.

### RELATED APPLICATION

This application is a continuation of U.S. application Ser. No. 10/687,396, filed Oct. 15, 2003, which is a continuation-in-part of, and claims the benefit of U.S. Ser. No. 10/376,538, filed Feb. 28, 2003, which claims priority of U.S. Ser. No. 60/360,758, filed Mar. 1, 2002. The entire teachings of the the above application(s) are incorporated herein by reference.

The invention described herein was made in the course of work under Department of Defense Breast Cancer grant numbers BC980415 and DAMD 17-99-1-9363. Accordingly, the United States government has certain rights in this invention.

### BACKGROUND OF THE INVENTION

### Cellular Signal Transduction and Protein Tyrosine Kinases/Protein Tyrosine Phosphatases

A wide variety of cellular signaling pathways sense and respond to changes in a cell's extra-cellular and intra-cellular environment, often regulating a cell's decision to proliferate, migrate, differentiate or self-destruct (called apoptosis). Overly activated or aberrantly regulated signaling pathways are a common occurence and important driving force in proliferative diseases, most notably cancer. A key component of most of these pathways are protein-tyrosine kinases (PTKs) and protein-tyrosine phosphatases (PTPases). PTKs are enzymes that modify cellular proteins by replacing selected tyrosine hydroxyl groups with covalently-linked phosphate groups. PTPases are enzymes that perform the opposite function, removing phosphate groups from protein-tyrosine residues and reforming the hydroxyl group on the tyrosine ring structure. The hundreds of cellular PTKs are broadly divided into two classes: receptor-tyrosine kinases, exemplified by growth-factor receptors such as the epidermal growth-factor receptor (EGFR), Her2/neu-ErbB2, and c-Met, and the non-receptor tyrosine kinases, exemplified by the Src family of PTKs (1).

Receptor tyrosine kinases (RTKs) are activated when they bind their growth factor ligand or dimerize. Once activated, the RTK phosphorylates itself on specific tyrosine residues that then serve as docking sites for the SH2 and phosphotyrosine-binding (PTB) domains of many second messenger proteins. Several of these proteins are then themselves tyrosine phosphorylated and thereby activated, propagating signaling cascades within the cell (2-4). One of these second messengers is the adapter protein Shc, which, when tyrosine phosphorylated, appears capable of signaling to several different pathways, discussed further below (5, 6). In perhaps the best characterized cascade, tyrosine phosphorylated (PY) Shc is recognized by Grb2-SOS complexes. As a result, SOS is translocated to the cellular membrane, which facilitates its ability to activate Ras (7-14). Ras then activates a highly regulated cascade through the Raf and MEK kinases, to the MAP kinases, ERK-1 and 2. Active Erk-1/2 in turn up-regulates a number of pathways required for the initiation of DNA synthesis end cell proliferation (34, 35).

### Aberrantly Active PTKs in Proliferative Diseases

Proliferative diseases such as psoriasis and cancer often have aberrantly high levels of PTK activity. In cancer cells the PTK activity is sometimes due to gross over-expression of the normal PTK (one example of which would be the over-expression of Her2 in 20% to 30% of women with invasive breast cancer), sometimes due to activating genetic changes in the PTK gene (point mutations, deletions of regulatory domains, or formation of fusion proteins stemming from chromosomal translocations, one example of which is the Bcr-Abl PTK in chronic myelogenous leukemia), and other times due to autocrine or paracrine activation or transactivation. In numerous rodent tumor models, the aberrantly activated PTKs have been shown to play key roles in tumorigenesis, tumorgenicity, and the tumor's malignant phenotype. In chronic myelogenous leukemia, it is clear from studies in rodent models that Bcr-Abl PTK is both responsible for the genesis of the disease and for the maintenance of its neoplastic properties. Consistent with this, Gleevec, a PTK inhibitor that targets the Bcr-Abl PTK, has had dramatic therapeutic effects in human patients in the chronic phase of this disease.

However, most cancers cannot be so simply characterized by and are not driven by a single, aberrantly active PTK. This is perhaps best exemplified by invasive breast cancer, a disease that is diagnosed in nearly 200,000 women in America and 1 million women world-wide yearly (15). Numerous growth factors and their receptors have been implicated in breast cancer development and aggressiveness (16-22). These include Her-2/neu, other members of the EGF-receptor family; hepatocyte growth factor (HGF) and its receptor, c-Met; IGF-1, IGF-II and the IGF-1 receptor; FGPs and their receptors; mammary-derived growth factor (MDGF-1 and its receptor); and non-receptor tyrosine kinases c-Src and Brk (reviewed in 23, and see 24, 25-27).

### The Need for a More Widely Applicable Mechanism-Based Molecular Therapeutic Target

Although, current therapies clearly improve relapse-free survival, about 20% of patients in the U.S. alone succumb to their disease, with this fraction being much higher worldwide (15). In an attempt to improve therapies, a major effort is being made to directly target specific growth-factor receptors. One of the first approaches having some success uses Herceptin, a humanized monoclonal antibody specific for Her2. However, in addition to its expense, its usefulness is limited to a subset of the 20% of breast cancers that over-express Her2. Many breast cancers may have other, aberrantly-activated PTKs on which they depend. However, it would be difficult to develop inhibitors specific for each individual receptor and non-receptor PTK that may be uniquely, or in combination, contributing to a single patient's tumor, to then determine which of these PTKs are likely to contribute to an individual tumor's refractoriness to traditional therapies, and then to treat them accordingly.

### Criteria Defining a Widely Useful Molecular Target

However, an alternative, broadly applicable strategy would be to target a signaling protein downstream in common to many different PTKs. Ideally, this signaling protein would be aberrantly regulated and play a critical role(s) in aggressive tumors that evade traditional surgical, radiotherapy and adjuvant therapy regimens: in other words, in tumors of patients who have a poor prognosis.

### Candidate Molecular Therapeutic Targets

There are a myriad of candidate target signaling proteins that are downstream of receptor PTKs, non-receptor PTKs, or both, proteins that include not only Shc, Grb2, SOS, Ras, Raf, MEK, the MAP kinases known as Erk1,2, but also phosphatidyl inosital 3′ kinase, protein kinase C, phospholipase C, SHP2, FRS2, CbI, Ship-1 and Ship-2, Grb10, Gab-1 and Gab-2, crk, rasGAP, and pl90racGAP, to name a few. Presently, it is not known which, if any, of these secondary signalling molecules fulfilled the requirements articulated above for a broadly useful molecular target.

### The Shc Proteins

The adapter protein, Shc, becomes tyrosine phosphorylated (PY) in response to signaling from all of these receptors, from non-receptor tyrosine kinases, from many G-protein-coupled receptors, and in response to cellular interactions with the extracellular matrix (6, also see 29, 30, 31). Shc is involved in responses to stimuli that activate cell proliferation, invasion, motility and control anchorage independent growth (4, 32-43). Further, several studies using microinjected antibodies to Shc, Shc antisense, and various Shc dominant-negative constructs have shown the dependence on a functional Shc of signaling from the EGF receptor, Her2/Neu, IGF-1 and HGF (5, 25, 44-46).

A single Shc gene in humans encodes the ubiquitous Shc A proteins, and two other, related genes encode the Shc B and C proteins that are found in cells of neuronal origin. The Shc A gene gives rise to the p52 and p46 Shc isoforms (4, 6, 47), and to a third isoform, p66, that contains a unique N-terminal domain (CH2) not found in p52 or p46 Shc. The synthesis of p66 Shc is driven from a separate promoter (6, 48). Although tyrosine phosphorylation of the p52 and p46 isoforms of Shc appears to drive these reactions forward, p66 Shc appears to inhibit some of these processes (49, 50); additionally, p66 Shc is an apoptotic sensitizer to oxidative stress (51, 52). Such stress may be generated by chronic activation of growth-factor pathways, by infiltrating neutrophils and macrophages, and by neovascularization of hypoxic tumors (53, 54).

### Evidence for a Wide Role for Shc in Cancer

A wealth of evidence implicates the Shc proteins in both breast cancer mouse models as well as in human breast cancer and prostate cancer. The appearance of multifocal aggressive mammary tumors in transgenic mice expressing either Polyoma virus middle T antigen or an oncogenic ErbB2 require the interaction of Shc with mT and ErbB2, respectively (55, 56). Previously it was noticed that most cell lines isolated from aggressive human breast cancers contained unusually large amounts of activated, PY-Shc (p52 and p46 Shc) but express little of the counterpoised p66 Shc (57). Further, it has been shown that PTK inhibitors that blunt the growth of human breast cancer cells in tissue culture also rapidly inhibit Shc tyrosine phosphorylation (57-59). Expression of a dominant-negative Shc (Y317F) point mutant inhibited the growth of the breast cancer cell lines that normally have high levels of PY-Shc, but did not inhibit the growth of normal breast epithelial cell lines (60). Similarly, it has been recently found that PC-3 prostatic carcinoma cells are no longer tumorgenic when the ability of their IGF-1 receptors to transactivate their EGFRs is blocked using a dominant-negative Shc (Y317F) point mutant (see below 61).

### SUMMARY OF THE INVENTION

This invention provides a method for treating a subject afflicted with a proliferative disorder comprising administering to the subject a therapeutically effective amount of an agent which inhibits the expression of p46 Shc and/or p52 Shc in the subject.

This invention further provides a method for treating a subject afflicted with a proliferative disorder comprising administering to the subject a therapeutically effective amount of an agent which inhibits the activity of p46 Shc and/or p52 Shc in the subject.

This invention further provides a method for treating a subject afflicted with a proliferative disorder comprising administering to the subject a therapeutically effective amount of an agent which increases the level of phosphorylated p66 Shc in the subject.

This invention further provides a method for inhibiting the expression of p46 Shc and/or p52 Shc in a cell comprising delivering to the cell an agent that inhibits the expression of p46 Shc and/or p52 Shc in the cell.

This invention further provides a method for inhibiting the activity of p46 Shc and/or p52 Shc in a cell comprising delivering to the cell an agent that inhibits the activity of p46 Shc and/or p52 Shc in the cell.

This invention further provides a method for increasing the level of phosphorylated p66 Shc in a cell comprising delivering to the cell an agent which increases the level of phosphorylated p66 Shc in the cell.

This invention further provides a method for determining whether an agent inhibits the phosphorylation of p46 Shc or p52 Shc comprising: (a) contacting p46 Shc or p52 Shc with the agent under conditions which, in the absence of the agent, would permit phosphorylation thereof; (b) determining the degree to which the p46 Shc or p52 Shc has deen phosphorylated; and (c) comparing the degree of phosphorylation measured in step (b) with the degree of phosphorylation measured in the absence of the agent, a greater degree of phosphorylation in the absence of the agent indicating that the agent inhibits phosphorylation of the p46 or p52 Shc.

This invention further provides a method for determining whether an agent inhibits the dephosphorylation of p66 Shc comprising: (a) contacting phosphorylated p66 Shc with the agent under conditions which, in the absence of the agent, would permit dephosphorylation thereof; (b) determining the degree to which the p66 Shc has been dephosphorylated; and (c) comparing the degree of dephosphorylation measured in step (b) with the degree of dephosphorylation measured in the absence of the agent, a greater degree of dephosphorylation in the absence of the agent indicating that the agent inhibits dephosphorylation of the p66 Shc.

This invention further provides a method for determining whether an agent inhibits the binding of a Shc A protein with a protein to which the Shc A protein must bind in a cell in order to carry out its proliferative function comprising: (a) contacting (i) the protein to which Shc A binds, or a Shc A-binding portion thereof, with (ii) Shc A or a suitable portion thereof, in the presence of the agent under conditions which, in the absence of the agent, permit binding; (b) determining the degree of binding; and (c) comparing the degree of binding measured in step (b) with the degree of binding measured in the absence of the agent, a greater degree of binding in the absence of the agent indicating that the agent inhibits the binding between the Shc A protein and the protein to which it must bind in a cell.

This invention further provides an article of manufacture comprising (a) a packaging material having therein an agent which inhibits the expression of p46 Shc and/or p52 Shc in a subject; and (b) a label indicating a use for the agent in treating a proliferative disorder in a subject.

Finally, this invention provides an article of manufacture comprising (a) a packaging material having therein an agent which inhibits the activity of p46 Shc and/or p52 Shc in a subject; and (b) a label indicating a use for the agent in treating a proliferative disorder in a subject.

### BRIEF DESCRIPTION OF THE FIGURES

FIG. 1. Forced Re-Expression of p66 Shc Inhibits Colony Formation on Soft Agar. Breast cancer cell lines (SKBR-3 and MDA-453) were transfected with p66-Shc expression plasmid, and multiple stable clones of each were obtained by antibiotic selection. Several clones of each were tested for their ability to form colonies on soft agar. Parental SKBR-3 and MDA-MB-453 cells and empty vector clones formed vigorous large colonies, whereas clones of SKBR-3 or MDA-453 cells re-expressing p66-Shc formed microcolonies or failed to grow at all. A representative parental 453 and a p66-clone are shown below.

FIG. 2. Relapse and survival as a function of p66 levels and adjuvent therapy. Scatter histogram of p66 Shc levels in patients' primary tumors as a function of vital and disease status at last follow-up. Patients' initial therapy either did not (Left Panel) or did (Right Panel) include systemic adjuvant.

FIG. 3. Constitutive expression of a wild-type p52 Shc-Gst fusion protein or a dominant-negative mutant p52 ShcY317F-Gst fusion protein in stably transfected PC-3 clones. Note inhibition of tyrosine phosphorylation of dn Shc in the phospho-tyrosine immunoblot (upper panel) despite equivalent total protein present in the total RaShc immunoblot (lower panel).

FIG. 4. Dominant negative Shc inhibits the tumorgenicity PC-3 clones in SCID-Beige mice. Eight-week-old, male SCID-Beige mice were subcutaneously implanted in the right rear flanks with either PC-3/wtShc, PC-3/dnShc, or PC-3 carrying the pEBG vector (107 cells/mouse). Tumor growth was monitored by external caliper measurements (n=4 mice/group; Bars, SEM).

FIG. 5. Scoring system for quantitating immunohistochemical staining intensity of PY-Shc and p66 Shc.

FIG. 6. Immunohistochemical staining of prostatic cancer specimens listed in Table 2. Sections were stained with the phospho-Shc specific antibody (anti-PY Shc) or phosphor-Erk specific antibody (anti-phospho-Erk), and counterstained with hematoxylin. Note that both the PY-Shc and phospho-Erk scores are very high in patient #4. However, the PY-Shc score is moderately high while the phospho-Erk score is zero in patient #5, consistent with our hypothesis.

FIG. 7. Immunohistochemical Staining of PY-Shc and p66 Shc in a Low-Shc Ratio, Non-recurring Prostate Tumor, and in a High-Shc Ratio, Recurring Tumor.

FIG. 8. The Shc Ratio Assay effectively dichotomizes early stage prostate cancer patients into those with a high risk and those with no risk of recurrent disease.

FIG. 9. Maps of hGST-Shc A (p52) and hshc A (p66).

### DETAILED DESCRIPTION OF THE INVENTION

Definitions:

“p52”, “P46” and “p66” shall refer respectively to the approximately 52-kDa, 46-kDa and 66-kDa Shc A proteins. The terms “p52”, “p52 Shc” and P52 Shc A” are used equivalently. Likewise, “p46”, “p46 Shc” and “p46 Shc A” are used equivalently, and “p66”, “p66 Shc” and “p66 Shc A” are used equivalently.

“Agent” shall include any organic or inorganic chemical. Examples of agents include amino acid, amino acid oligomer, amino acid polymer, natural or synthetic polypeptide or synthetic analog thereof, including phosphomimetic derivatives and dephosphomimetic derivatives thereof; any protein, including natural or recombinant or humanized antibodies or polypeptide or other ligands or analogs thereof that bind to cell surfact PTKs or that bind to cell surface receptors that activate PTKs; any natural product or chemical or enzymatic derivative or analog thereof; and any lipid or phospholipid; drug or medicinal compound. Further examples include tyrosine kinase inhibitors that inhibit the enzymatic function of tyrosine kinases, including without limitation, Gleevec (ST1571, Imatinab, cgp57148B), OSI-774, PP1, PP2, SU6656, SU4984, SU9518, SU5416, Genistein, Herbamycin A., PKC412, the tyrphostins (which include CI-1033, PD168393, PD513032, AG126, AG1478, AG879, AG957, ZM39923, ZM449829, Iressa, ZD1839, Gefitinib, Emodin, Erbstatin, B46, Quinazolones, and others), and tyrosine phosphatase inhibitors that inhibit the ability of tyrosine phosphatases to specifically cleave the phosphate moiety from tyrosine phosphate in protein. Further examples include agents that promote demethylation of the genetic promoter region that regulates transcription of mRNA for p66 Shc.

“Anti-proliferative agent” shall include any agent that attenuates any malignant property of a tumor, a tumor cell, another proliferative disease, or a cell associated therewith. An “anti-proliferative agent” may or may not inhibit the growth of tumor cells. Instead of, or in addition to inhibiting cell growth, (a) it may increase the likelihood of a tumor cell's undergoing apoptotic death, (b) it may inhibit the ability of a tumor cell to migrate, invade or metastasize, (c) it may inhibit the ability of a tumor cell to encourage its host to populate the tumor with new vasculature, (d) it may blunt the ability of a tumor to damage and remodel host tissue by inhibiting the elaboration and/or activation of extra-cellular degradative enzymes, and (e) it may increase the immunogenicity of a tumor cell, thereby activating tumor cell destruction by the host's immune system.

“PY-Shc” shall include the Shc A proteins phoshorylated on any or all of the tyrosine residues numbered Y239, Y240, and Y317 in the human p52 Shc, and the corresponding tyrosine residues in p46 and p66 Shc.

“Subject” shall mean any animal, such as a mammal, and shall include, without limitation, a mouse and a human.

### Embodiments of the Invention

A wealth of experimental evidence suggests critical roles for high levels of tyrosine-phosphorylated (PY) p52 and p46 Shc A proteins and low expression of p66 Shc A protein in breast, prostate, and other cancers. Considered together with the discovery that these Shc proteins have very strong prognostic ability in clinical breast and prostate cancer, tyrosine-phosphorylated Shc and p66 Shc meet the criteria for widely useful molecular mechanism-based therapeutic targets. Because unscheduled Shc activation and protein-tyrosine kinase (PTK) activation occur in many proliferative diseases, it is expected that targeting Shc proteins will be useful not only in breast and prostate cancer, but also in many other cancers and in proliferative diseases such as psoriasis.

The invention described here comprises compositions and methods for the development and identification of molecular agents that interfere with the functioning or amount of p46, p52, and/or p66 Shc A proteins.

The invention further comprises the use of agents that interfere with the functioning or amount of p46, p52, and/or p66 Shc A proteins for the therapeutic treatment of patients afflicted with breast cancer, prostate cancer, other cancers, and proliferative diseases such as psoriasis.

Specifically, this invention provides a method for treating a subject afflicted with a proliferative disorder comprising administering to the subject a therapeutically effective amount of an agent which inhibits the expression of p46 Shc and/or p52 Shc in the subject.

This invention further provides a method for treating a subject afflicted with a proliferative disorder comprising administering to the subject a therapeutically effective amount of an agent which inhibits the activity of p46 She and/or p52 Shc in the subject.

This invention further provides a method for treating a subject afflicted with a proliferative disorder comprising administering to the subject a therapeutically effective amount of an agent which increases the level of phosphorylated p66 Shc in the subject.

This invention further provides a method for inhibiting the expression of p46 Shc and/or p52 Shc in a cell comprising delivering to the cell an agent that inhibits the expression of p46 Shc and/or p52 Shc in the cell.

This invention further provides a method for inhibiting the activity of p46 Shc and/or p52 Shc in a cell comprising delivering to the cell an agent that inhibits the activity of p46 Shc and/or p52 Shc in the cell.

This invention further provides a method for increasing the level of phosphorylated p66 Shc in a cell comprising delivering to the cell an agent which increases the level of phosphorylated p66 Shc in the cell.

In one embodiment of the instant methods the agent is selected from the group consisting siRNA, a ribozyme, or a DNAzyme. In this invention molecular biological methods for altering Shc expression or function include, but are not limited to, reducing p46 and/or p52 Shc expression using siRNA approaches well known to those skilled in the art; constructing end expressing “dominant negative” mutants of the Shc A proteins or isolated sub-regions or derivatives or analogs of sub-regions of the Shc A proteins; constructing and expressing “dominant active” mutants of the Shc A proteins (especially p66 Shc) or isolated sub-regions or derivatives, or analogs of sub-regions of the Shc A proteins.

In another embodiment of the instant methods, the agent specifically inhibits the dephosphorylation of the Ser36 residue of phosphorylated p66 Shc in the subject. In another embodiment of the instant methods, the agent is a p66 Shc-encoding expression vector. In another embodiment of the instant methods, the subject is human. In one embodiment of the instant methods, the proliferative disease is prostate cancer, ovarian cancer or breast cancer. In another embodiment of the instant methods, the cell is a prostate cancer cell, an ovarian cancer cell or a breast cancer cell.

In this invention, administering agents can be effected or performed using any of the various methods and delivery systems known to those skilled in the art. The administering can be performed, for example, intravenously, orally, nasally, via implant, transmucosally, transdermally, intramuscularly, and subcutaneously. The following delivery systems, which employ a number of routinely used pharmaceutical carriers, are only representative of the many embodiments envisioned for administering agents in the instant invention.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E end C, and ascorbic acid), anti-caking agents, coating agents, and chelating agents (e.g., EDTA).

Determining effective amounts of agents for use in the instant invention can be done based on animal data using routine computational methods.

This invention further provides a method for determining whether an agent inhibits the phosphorylation of p46 Shc or p52 Shc comprising: (a) contacting p46 Shc or p52 Shc with the agent under conditions which, in the absence of the agent, would permit phosphorylation thereof; (b) determining the degree to which the p46 Shc or p52 Shc has been phosphorylated; and (c) comparing the degree of phosphorylation measured in step (b) with the degree of phosphorylation measured in the absence of the agent, a greater degree of phosphorylation in the absence of the agent indicating that the agent inhibits phosphorylation of the p46 or p52 Shc.

This invention further provides a method for determining whether an agent inhibits the dephosphorylation of p66 Shc comprising: (a) contacting phosphorylated p66 Shc with the agent under conditions which, in the absence of the agent, would permit dephosphorylation thereof; (b) determining the degree to which the p66 Shc has been dephosphorylated; and (c) comparing the degree of dephosphorylation measured in step (b) with the degree of dephosphorylation measured in the absence of the agent, a greater degree of dephosphorylation in the absence of the agent indicating that the agent inhibits dephosphorylation of the p66 Shc.

This invention further provides a method for determining whether an agent inhibits the binding of a Shc A protein with a protein to which the Shc A protein must bind in a cell in order to carry out its proliferative function comprising: (a) contacting (i) the protein to which Shc A binds, or a Shc A-binding portion thereof, with (ii) Shc A or a suitable portion thereof, in the presence of the agent under conditions which, in the absence of the agent, permit binding; (b) determining the degree of binding; and (c) comparing the degree of binding measured in step (b) with the degree of binding measured in the absence of the agent, a greater degree of binding in the absence of the agent indicating that the agent inhibits the binding between the Shc A protein and the protein to which it must bind in a cell.

This invention further provides an article of manufacture comprising (a) a packaging material having therein an agent which inhibits the expression of p46 Shc and/or p52 Shc in a subject; and (b) a label indicating a use for the agent in treating a proliferative disorder in a subject.

This invention further provides an article of manufacture comprising (a) a packaging material having therein an agent which inhibits the activity of p46 Shc and/or p52 Shc in a subject; and (b) a label indicating a use for the agent in treating a proliferative disorder in a subject.

Finally, this invention provides an article of manufacture comprising (a) a packaging material having therein an agent which increases the level of phosphorylated p66 Shc in a subject; and (b) a label indicating a use for the agent in treating a proliferative disorder in a subject.

This invention is illustrated in the Examples section which follows. This section is set forth to aid in an understanding of the invention but is not intended to, and should not be construed to limit in any way the invention as set forth in the claims which follow thereafter.

### EXAMPLES

### Introduction

This invention is based on the novel discovery in the primary tumors of patients with breast or prostate cancer that high levels of tyrosine-phosphorylated (PY) Shc A proteins, herein designated p46 and p52 Shc (although p66 Shc may also be tyrosine-phosphorylated), and low expression of the inhibitory Shc A isoform, herein designated p66 Shc, identify those patients who have a high likelihood of failing their primary therapy. This novel clinical finding, coupled with the mounting experimental evidence for the mechanistic involvement of the Shc proteins in many cellular processes associated with aggressive tumors, satisfied the criteria for candidate molecular targets that should be widely effective therapeutic targets in invasive breast cancer, prostate cancer, as well as in many other types of cancers and proliferative diseases such as psoriasis.

This invention identifies the p42, p56, and p66 Shc A proteins as likely widely useful targets in proliferative diseases. This invention further identifies the p42, p56, and p66 Shc A proteins as likely widely useful targets in the proliferative diseases: invasive breast cancer, prostate cancer, and many other cancers, including but not limited to ovarian cancer, cancer of the gastrointestinal tract, head and neck cancer, thyroid cancer, glioblastoma, melanoma, and basal cell carcinoma; and in the proliferative diseases including, but not limited to psoriasis.

The invention described here comprises compositions and methods for the development and identification of molecular agents that interfere with the functioning or amount of p46, p52, and/or p66 Shc A proteins in cells. The invention further comprises the use of agents that interfere with the functioning or amount of p45, p52, and/or p66 Shc A proteins for the therapeutic treatment of patients afflicted with breast cancer, prostate cancer, other cancers, and proliferative diseases such as psoriasis.

### EXAMPLE 1

### Experimentals

### P66 Shc Inhibits Anchorage-Independent Growth of Breast Cancer Cells

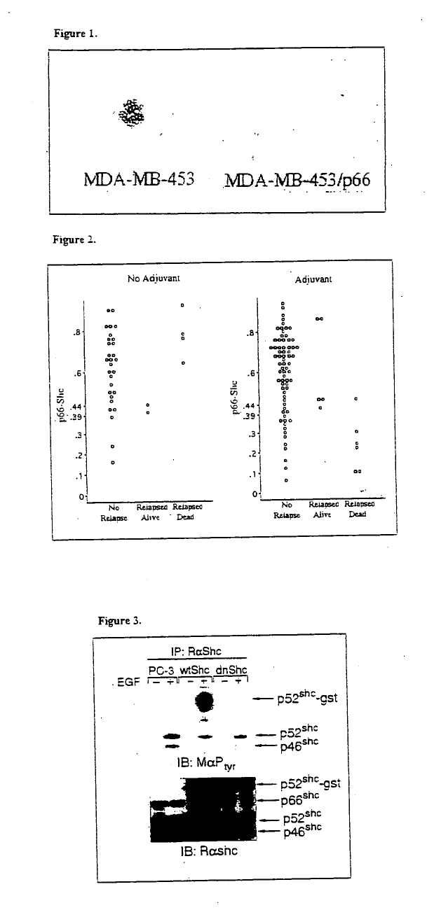

Previously only low levels of p66 Shc had been detected in breast cancer cells that express high levels of PY-Shc and appear to depend upon PY-Shc signaling for various aspects of their neoplastic phenotype. This suggested that p66 Shc might normally suppress tumor development, and that loss of p66 Shc expression might convey selective advantage to tumor cells. To test this notion, the SKBR-3 and MDA-MB-5453 breast cancer cell lines that express very little, if any, p66 Shc were utilized. By transfecting these cells with a p66-Shc expression vector, several independent clones of SKBR-3 and MDA-MB-453 that re-expressed normal levels of p66 Shc were generated. Although these cells grew well in tissue culture, they lost ability to form colonies in soft agar, a classic anchorage-independent growth test that correlates with tumorgenicity (FIG. 1).

Of particular potential relevance to likely mechanisms whereby only tumor cells with activated Shc seem able to elude the surgeon's scalpel (see data below, and 28)), activated Shc plays an important role in cell migration (42, 43, 62), and it's interaction with beta4-integrin is requisite for beta4-integrin and c-Met-mediated cell invasion (45, 63). Furthermore, PTEN, a tumor suppressor implicated in the high incidence of breast cancer in patients with Cowden's disease, dephosphorylates and thereby inactivates Shc (43, 64, 65).

### Prognostic Value of PY-Shc and p66 Shc in Breast Cancer

Considered together, all of this information suggests that high amounts of PY-Shc relative to the 66-kDa Shc isoform would serve as a marker for aggressive neoplasms. Semi-quantitative immunohistochemical analyses of PY-Shc and p66 Shc were performed on archival primary breast tumor specimens from 116 women, 17 of whom experienced relapse (6.1 years median follow-up of non-relapsed patients). Consistent with our hypothesis, staining intensities demonstrated that increased amounts of PY-Shc (P=0.01) and decreased expression of p66-Shc protein (P=0.028) correlated with disease recurrence. Modeled as the ratio of PY-Shc to p66 Shc, the Shc Ratio correlated strongly with nodal status (P=0.003), tumor stage (P=0.0025) and disease Stage (P=0.002), and was 2-fold higher in primary tumors of patients who subsequently relapsed (P<0.001). Univeriate Cox proportional hazards analysis of relapse-free survival demonstrated the prognostic value of PY-Shc (P=0.01), p66-Shc (P=0.04) and the Shc Ratio (P=0.004) as continuous variables, with a Hazard Ratio (HR) of 10 (P=0.007) for the Shc Ratio. Shc Ratio cut points of <0.35 and >0.65 were identified and independently validated to maximize negative predictive value and positive predictive value. Patients with low Shc-Ratios (n=36) had 0.08 HR of relapse (P=0.007) compared to patients with high Shc Ratios, experiencing an 8-year cumulative 2.9%, and 55% relapse hazard, respectively, compared to a 22% relapse hazard in the total cohort. The Shc Ratio had similar prognostic value for disease-specific survival. In multivariate models, the Shc Ratio, both as a continuous variable and as a cut point-categorized variable, was independent of all measured covariates, including nodal status, tumor stage, disease stage, grade, estrogen-receptor status and adjuvant therapy, and was a stronger prognostic marker than all but nodal status. All relapsed node-positive patients had very high Shc Ratios (>0.80, P=0.006) in their primary tumors. Furthermore, the Shc Ratio was a strong, independent prognostic indicator in node-negative patients (79 patients, 10 recurrences), with a HR of 0.086 (P=0.02) that was independent of clinical markers and adjuvant therapy. Patients with Low and High Shc Ratios experienced a 3.6% and 64% relapse hazard, respectively, compared to 20% in the total node-negative cohort.

### Shc Ratio: Prognostic or Predictive? Effects of Therapy

There were no significant differences in therapy received by patients in the low, intermediate or high Shc-Ratio groups (P=0.53), suggesting that therapeutic differences did not confound assessment of the prognostic value of the Shc variables. Consistent with this, the Shc variables retained their full independent prognostic value when adjusted for therapy in a bivariate Cox proportional hazards model or stratified model, and in a multivariate model, therapy approached significance, but did not alter the prognostic value of the Shc Ratio either as a categorical or continuous variable.

Useful markers may be prognostic independent of therapy, predictive of response to therapy, or a mixture of both (66). For breast cancer, it would be important to know if a marker can accurately assign risk to patients whose only treatment has been removal of the primary tumor: a small cohort of 25 patients met this criteria. Even though the Shc Ratios of these patients were very evenly distributed (9 low, 8 intermediate and 8 high), all three patients who relapsed had high Shc Ratios; all had Stage I disease. By log rank analysis, the categorical Shc Ratio was a significant predictor of relapse (P=0.018), and by likelihood ratio (LR) analysis of Cox regression, the Shc Ratio as a continuous variable also had significant prognostic strength (X²=4.74 degrees of freedom (d.f.)=1, P=0.029). By Nelson-Aalen analysis, the 7 year cumulative relapse hazard of patients with high Shc Ratios was 0.77. Thus, although this is a very small group of patients, their data suggest strongly that the Shc Ratio has prognostic value in the absence of systemic adjuvant therapy.

However, the large majority of patients (81 patients; 11 relapses) received systemic adjuvant treatment. Again, though the Shc Ratios were rather evenly distributed (24 low, 23 intermediate and 34 high), 9 of the 11 relapses occurred in patients with high Shc Ratios; none in patients with low Shc Ratios (Cox regression LR X²=11.87, d.f.=2, P=0.0027 for the Shc Ratio as a categorical variable; P=0.004; LR X²=5.78, d.f.=1, P=0.016 for the Shc Ratio as continuous variable) (28). Thus, the Shc Ratio was clearly prognostic in the presence of systemic adjuvant treatment as well. However, for adjuvant-treated patients with high Shc Ratios, the 7 year cumulative relapse hazard by Nelson-Aalen analysis was only 0.33, considerably less than the 0.77 seen in the small cohort receiving only surgical therapy. If this difference is maintained in a large, well-controlled study, it would suggest that the high Shc Ratio identifies patients who respond well to systemic adjuvant therapy.

TABLE 1
Cox proportional hazards anlysis of RFS and
DSS for Shc variables in breast cancer

| Shc | RFS^{a} | | DSS^{a} | |
|---|---|---|---|---|
| Variables | HR (95% CI) | P | HR (95% CI) | P |

Continuous Variables

| | | | | | | |
|---|---|---|---|---|---|---|
| PY-Shc | 8.14 | (1.8-37) | 0.013 | 2.50 | (0.35-18) | 0.21 |
| P66 Shc | 5.36 | (1.2-25) | 0.035 | 9.71 | (1.3-71) | 0.031 |
| Shc-Ratio | 10.3 | (2.5-42) | 0.004 | 14.0 | (2.6-76) | 0.005 |
| PY-Shc^{b} | 13.9 | (2.6-75) | 0.005 | 3.5 | (0.42-29) | 0.16 |
| P66-Shc^{b} | 7.69 | (1.7-34) | 0.013 | 10.6 | (1.5-76) | 0.043 |

Categonical Variable
Shc Ratio
cut points^{c}

| | | | | | | |
|---|---|---|---|---|---|---|
| Low vs High | 0.078 | (0.014-0.429) | 0.007 | 0.160 | (0.028-0.93) | 0.044 |
| Intermediate | 0.246 | (0.086-0.706) | 0.015 | 0.337 | (0.090-1.26) | 0.088 |
| vs High | | | | | | |
| High | 1.0 | | — | 1.0 | | — |

^{a}HR for an increase in Py-Shc or in the Ratio of Py-Shc to p66 Shc, or for a decrease in p66 Shc, each equal to the full observed scoring range of these continuous variables. Hazards for the Low and Intermediate Shc Ratio groups were calculated relative to the high-Shc Ratio subgroup. HR for High relative to Low Shc Ratio groups is the inverse of the Low to High HR, or 12.8 for RFS. Wald-test values for P < 0.05 are bolded.
^{b}Bivariate Cox analysis mutually adjusted for PY-Shc and p66-Shc as covariates.
^{c}Shc Ratio cutpoints: Low, <0.35; Intermediate, ≧0.35 and ≦0.65; High >0.65.

By LR analysis of bivariate Cox models mutually adjusted for PY-Shc and p66 Shc, high values of PY-Shc were primarily responsible for identifying patients who relapsed but survived (LR ΔX²=7.31, Δd.f.=1, P=0.0068 compared to p66 LR ΔX²=1.21, Δd.f.=1, P=0.27).

In contrast, low values of p66 Shc predicted relapse followed by death from disease (compare the Wald-test P values in Table 1). Strikingly, the primary tumors of all patients who did not receive primary adjuvant therapy and died of their disease expressed high levels of p66 Shc, while primary tumors of patients who did receive primary adjuvant therapy and died of their disease expressed low levels of p66 Shc (FIG. 2).

By Cox proportional hazards analysis of adjuvant-treated patients, low expression of p66 Shc was an extremely strong predictor of death from their disease (HR 585, LR X²=13.56, Δd.f.=1, P=0.0002). Arguing against a selection bias regarding adjuvant therapy, there was no difference in the p66 levels of non-relapsed patients comparing those patients who did, with those who did not receive systemic adjuvant (means of 0.57±0.02 SE and 0.62±0.03 SE, respectively, P=0.2). This provocative observation suggests further that therapeutically targeting both PY-Shc and p66 Shc might be especially efficacious.

### Evidence for a Role of Activated-Shc in Prostate Cancer Progression

To address functional roles of Shc in prostate cancer receptor activation of Shc by immunoprecipitation/immunoblot analysis of several prostate cancer cell lines including androgen-sensitive LnCaP cells, and androgen-insensitive DU-145 and PC-3 cells has been previously assessed. Consistent with previous reports (67), Py-Shc was detected in all three cell lines upon exogenous stimulation with EGF. In parallel, immunocytochemical analysis of DU145 and PC-3 using our rabbit polyclonal antibodies specific for Shc phosphorylated on tyrosine residue 317 (anti-PY-Shc) demonstrated a dramatic increase in antibody-immunolabeling in the presence of EGF stimulation.

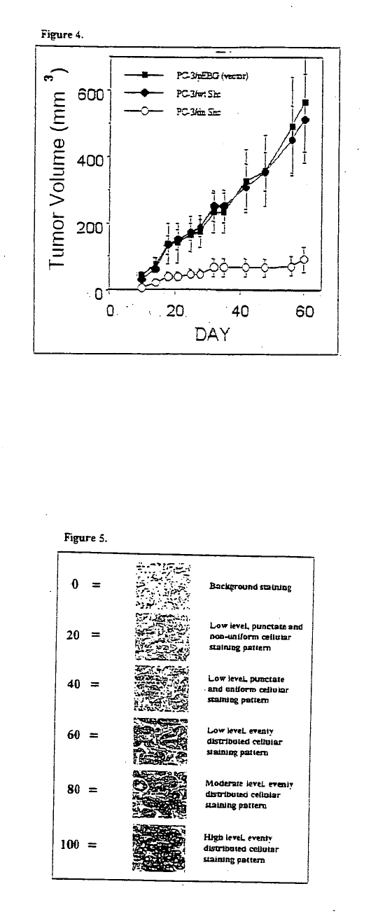

Next, PC-3 cells were stably transfected to constitutively express wild-type (wt) Shc (p52) or a dominant-negative (dn) mutant p52 Shc (Y317F-Shc) in which Tyr317 has been mutated to a phenylalanine thus preventing tyrosine phosphorylation at this site and thereby inhibiting interaction with Grb2. Expression of wtShc and dnShc was confirmed in these cells; as expected, EGF stimulated little tyrosine phosphorylation of the dn-Shc protein (presumably only on the other tyrosine phosphorylation sites at residues 239, 240) (FIG. 3). Because of the reported dependence of PC-3 cells upon autocrine IGF-1 receptor stimulation for growth in serum free media (68), and because it had been recently demonstrated that IGF-1 signaling is dependent upon Shc in 3T3L-1 preadipocytes (69), the wtShc cells and dnShc cells shown in FIG. 3 were also examined for their ability to proliferate in serum-free media. As predicted, the dnShc cells were unable to proliferate in serum-free medium compared to parental PC-3 cells and wtShc cells. When wtShc cells, dnShc cells, and PC-3 cells containing the pEBG vector were implanted into male Severe-combined-immunodeficient (SCID)-Beige mice, wtShc tumors and vector tumors demonstrated comparable growth, but dnShc tumors were strongly growth inhibited (FIG. 4).

Immunoprecipitation of Shc protein from these tumors, grown for 60-days in mice, demonstrated that tumor cells continue to express wtShc or dnShc in vivo (not shown).

When wtShc cells and dnShc cells were examined for ability of growth factors to stimulate downstream activation of Erk-1/2, dnShc cells demonstrated attenuated singaling in response to EGF, FGF, and IGF compared to parental cells and wtShc cells. Considered together, these findings strengthened our rationale for assessing phosphorylated-Shc as a biological marker for prostate cancer progression.

### Preliminary Immunohistochemical Evaluation of Prostatic Carcinoma

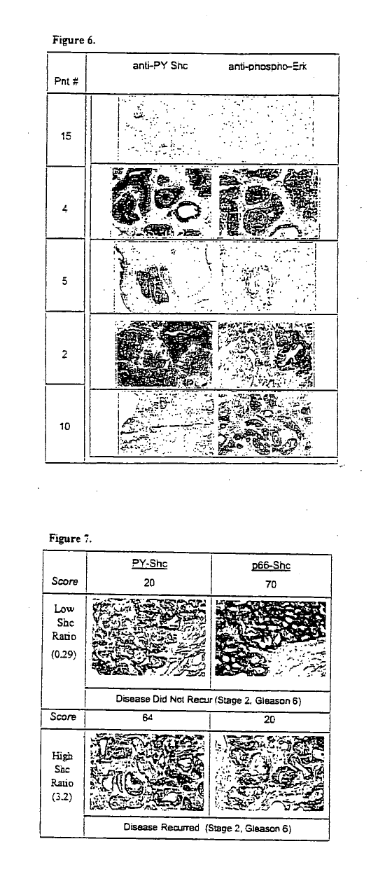

The current visual scoring system for stain intensity uses a 0, 20, 40, 60, 80, 100 scale (FIG. 5). The 0-100 staining intensity is multiplied by the fraction of the tumor in the entire specimen that stains at each intensity level. These are then summed, thus arriving at a total average intensity (0-100 scale) of staining for all of the tumor tissue in the secton.

TABLE 2
Immuno-histochemical staining for PY-Shc and phospho-Erk
in prostate biopsy specimens. Patients 1-10, adenocarcinoma.
Patient 15, normal prostate.

| Patient | PY-Shc Score | pERK Score | PSA | Gleason |
|---|---|---|---|---|
| 1 | 65 | 00 | High | 7 |
| 2 | 90 | 40 | 7.2 | 7 |
| 3 | 65 | 70 | 6.9 | 6 |
| 4 | 60 | 60 | 4.8 | 6 |
| 5 | 35 | 00 | 19.0 | 6 |
| 6 | 20 | 00 | 4.9 | 6 |
| 7 | 15 | 15 | 1.1 | 7 |
| 8 | 55 | 65 | 5.4 | 6 |
| 9 | 65 | 55 | 11.8 | 7 |
| 10 | 20 | 50 | High | 6 |
| 15 | 0 | 0 | 5.2 | 0 |

Initally biopsy specimens of 10 patients recently diagnosed with prostatic carcinoma, as well as several specimens showing no pathology, were evaluated for staining with anti-PY-Shc and anti-phosphoErk, and compared these values with PSA levels and Gleason score (see examples in FIG. 6 and Table 2). Note in particular that patient #5, and less markedly, patient #2 demonstrated strong PY-Shc staining but absent or relatively weak phospho-Erk staining. This observation is not surprising, for there are many examples emerging where growth-factor receptors and Shc are activated but Erk-1/2 are not (33, 70, 71). The wide range of intensity and density of PY-Shc staining in even this small sample with Gleason scores restricted to 6 and 7 demonstrated that the proposed study was technically feasible and likely to yield clear results.

### Preliminary Evaluation of the Shc Ratio Test as a Prognostic Marker in Early Stage, Organ-Confined Prostate Cancer

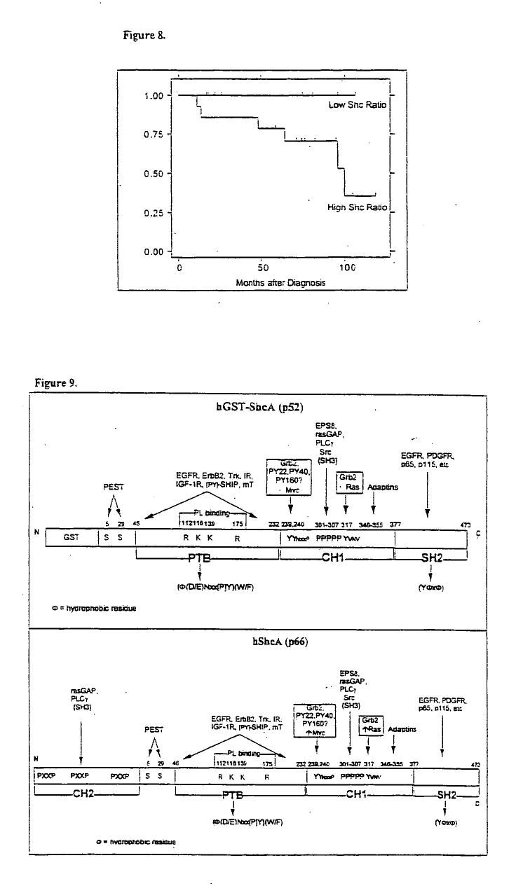

Archival primary tumor specimens from 22 previously untreated patients with early stage prostate cancer were next analyzed. Vicinal tumor-containing sections were immuno-stained for PY-Shc and p66 Shc, scored (blinded to patient identity, clinical characteristics and outcome) and dichotomized into those with a ratio of PY-Shc to p66-Shc of >0.6 (High Shc Ratio), and into those with a ratio of Py-Shc to p66-Shc of <=0.6 (Low Shc Ratio) (see example in FIG. 7) the same She Ratio cutoff that had been used successfully in breast cancer. Of the 22 patients, 6 patients had experienced recurrent disease: all 6 had High Shc Ratios. None of the 8 patients with Low Shc Ratios experienced recurrent disease (see FIG. 8).

As with breast cancer, the Nelson-Aalen cumulative risk function is consistent with eventual disease recurrence in nearly all 14 of the High Shc Ratio patients. Even in this very small preliminary group, the dichotomized Shc Ratio had significant prognostic value by Cox Proportional Hazards analysis (P=0.03). Fisher's Exact (two-sided) test estimates statistical significance at p=0.05. Yet, notwithstanding the group's small size, the clinical significance and implications of these preliminary data are striking. The patients with recurring disease included one AJCC stage 1 (Gleason 6), one AJCC stage 1 (Gleason 8), and four AJCC stage 2 disease (Gleason scores of 5, 6, 6, and 7). The mean follow-up was 5.8 years for non-recurring patients, with a mean time to disease recurrence of 4.2 years in the recurring patients. These data compare favorably with a recent preliminary report using gene expression profiling as a prognostic indicator in prostate cancer (72), especially considering that the report included patients with advanced stage and high Gleason score.

Synopsis

In summary, all of the experimental evidence recited and detailed above implicate Shc proteins in breast and prostate cancer. Considered together with our novel discovery that the Shc proteins have very strong prognostic ability in clinical breast and prostate cancer, Py-Shc and p66 Shc meet the criteria for widely useful molecular mechanism-based therapeutic targets. Because unscheduled PTK activation occurs in many proliferative diseases, it is expected that targeting Shc proteins will be useful not only in breast and prostate cancer, but also in many other cancers and in proliferative diseases such as psoriasis.

### Functional Structure of the p46, p52, and p66 Shc A Proteins

The p46 and p52 Shc proteins are approximately 46 kDa and 52 kDa, respectively. They are comprised of a N-terminal phosphotyrosine-binding domain (PTB), a central CH1 domain and a C-terminal SH2 domain (shown in FIG. 9 with an added glutathione-S-transferase fusion tag on the N-terminus). The p46 Shc, synthesized from an alternative translational initiation site on the p52 Shc mRNA, lacks a short N-terminal sequence that interacts with PEST PTPase when P52 ShcS (29) is phosphorylated (28). This region also appears to markedly increase the affinity of the PTP domain for specific tyrosine-phosphorylated motifs (29). The PTB domain can bind to specific phosphotyrosyl residues in the EGF receptor, Her2/ErbB2, the insulin receptor, polyoma middle T antigen, to name a few. The PTB domain also contains a lipophilic region homologous to pleckstrin, and appears to function in helping to localize a sub-population of Shc to the cell membrane. The SH2 domain recognizes a different phosphotyrosyl motif than the PTB domain (see FIG. 9). The Shc SH2 domain binds to other specific phosphotyrosyl residues on the EGF receptor, the PDGF receptor, and other cellular proteins. The CH1 domain contains the Y(239), Y(240) and Y(317) tyrosine phosphorylation sites. The Y(239), Y(240) sites appear to be preferentially phosphorylated by the non-receptor Src-family of PTKs, while Y(317) appears to be targeted preferentially by receptor-type PTKs. Both the Y(239) and Y(240) sites serve as high-affinity docking sites for Grb2, although at least in some systems, the Y(239) site appears to signal to Myc, not the Erks. The Y(317) reportedly interacts with Grb-2-Gab2 complexes in signaling to P13 kinase. The CHI domain also contains PxxP motifs (a.a. 301-307) that characteristically interact with SH3 protein domains. This motif in the CH1 domain reportedly binds to the SH3 domains of: the Src family of PTKs, PLC, rasGAP and EPS8, among others. The Shc protein also provides survival signals, and can up regulate Bcl-2. Shc also plays important roles in cellular interactions with extracellular matrix and the cytoskeleton, interacting with focal-adhesion kinase (FAK), integrins and CEA-CAM.

The p66 Shc mRNA transcription is driven by an alternative promoter, and in addition to encoding the p52 and p46 translational start sites, p66 MRNA codes for an additional 110 amino acid N-terminal domain named CH2 (FIG. 9). While p52 and p46 are typically expressed in relatively invariant amounts, p66 Shc expression appears down regulated in most cells of hematopoetic lineages and in aggressive breast and prostate cancers (detailed above), and in other cancers as well. The down regulated expression of p66 Shc appears to be due in part to hypermethylation of the 66 Shc's unique promoter. Serine (36) in the CH2 domain appears to be phosphorylated in response to MEK activation, thereby complexing with Grb2 in a non-productive manner (at least in terms of Ras activation), and also is phosphorylated in a non-MEK-dependent manner in response to oxidative stress. As a result, Akt/PKB is activated and in turn phosphorylates a Forkhead transcription factor, preventing it from entering the nucleus. This particular Forkhead transcription factor would otherwise stimulate catalase mRNA production, up-regulating cellular catalase which could then the reactive hydrogen peroxide species. The p66 Shc, thus, blocks this protective response to oxidative stress, and thereby acts as an apoptotic sensitizer.

### Example 2

### Additional Embodiments

### Proliferative Assays

One embodiment of the instant method identifies anti-proliferative agents based on their ability to alter the function or cellular levels of the Shc A proteins. In one version of this method, the candidate agents are contacted with one or more indicator cell lines in tissue culture for a sufficient period of time to allow the agent to act on the cells and alter Shc functions or amounts; which cell lines may include, but are not limited to the breast cancer cell lines known as SKBR3, BT474, MDA-MB-453, MDA-MB-468, MDA-MB-361, ZR-75-1, T47-D, and MCF-7. Alteration of Shc function or level can be detected and quantitated by any of a number of methods familiar to those skilled in the art. These methods include but are not limited to:

(a) The level of phosphorylated tyrosine 239, 240, and/or 317 in Shc A proteins can be semi-quantitatively determined by:

(a) (i) fixing the cells in formalin and reacting the fixed cells with antibodies respectively specific for PY[239]-Shc, PY[240]-Shc, PY[239,240]-Shc and PY[317]-Shc; and then washing away the unbound antibodies and detecting the bound antibodies using any of a number of methods familiar to those skilled in the art, in such methods as we have described in a publication in press (28), methods using direct, indirect antibody systems wherein one or more of the detecting components are labeled with a radioactive tracer or fluorphore, or an enzyme such as alkaline phosphatase or horse-radish peroxidase; and where the presence of bound alkaline phosphatase or horse-radish peroxidase is detected by a precipitating or soluble chromogenic or fluorogenic substrate, either directly or by an amplified system such as the tyramide-based systems. The presence of the bound radioactive label can be detected and quantitated by a number of means familiar to those skilled in the art; the soluble chromophore can be quantitated spectrophotometrically; the soluble fluorophore can be quantitated in a fluorometer; the precipitated chromophore and fluorophore can be detected and semi-quantitated using light and fluorescence microscopy, respectively.

(a) (ii) extracting the cells in a detergent cocktail such as Laemmli sample buffer, separating the extracted proteins by reducing SDS PAGE, transferring the resolved proteins directly to a supporting matrix such as.a nitrocellulose filter and then detecting the presence of PY-Shc proteins by probing the filter with each (combined or separately) of the afore-mentioned PY-Shc-specific antibodies, and quantitating the amount of bound antibody using radiolabels or alkaline phosphatase or horse-radish peroxidase-conjugates, and quantitating bound antibody by techniques familiar to those skilled in the art, including chemiluminescence.

(a) (iii) extracting the cells with a detergent-buffer cocktail containing 1% Triton X-100, inhibitors of kinases, phosphatases, and a slightly basic buffer such as phosphate or Tris at pH near 7.4 (see 57) or a detergent-buffer cocktail containing stronger detergents such as 0.1% SDS and 0.5% sodium deoxycholate; then immunoprecipitating the Shc proteins using either the afore-mentioned antibidoes specific for PY-Shc, antibodies to PY, or antibodies to the Shc protein structure, then resolving the immunoprecipitated proteins on SDS PAGE, transferring the resolved proteins to filters, and then probing with antibodies directed as appropriate to PY, PY-Shc or the Shc protein structure and detecting as described in (a) (ii), above.

(a) (iv) Extract cells as in (a) (iii) except quantitate PY-Shc using a sandwich-type ELISA with either anti-Shc protein or anti-PY-Shc covalently linked to a the solid phase, contacting the extract with the solid phase and then probing with the complementary antibody (anti-PY-Shc or anti-Shc protein, respectively) and then quantitating the bound probing antibody as in (a) (ii), except that the solid phase and probing antibody must either be sufficiently different (e.g., mouse and rabbit) so as to be separately detectable, or the probing antibody must be directly tagged with either the detecting enzyme or with biotin or with another similar moiety that can be specifically recognized by an appropriate antibody, other protein, or reagent.

(b) The level of Shc proteins can be quantitated using assays analogous to the assays described in paragraph (a), using anti-Shc protein (either reactive with all Shc A isoforms, for example antibody directed to domains or regions shared by all of the isoforms; or reactive only with epitopes unique to the CH2 domain, which is unique to p66 Shc A).

(c) The level of p66 ShcS[36P] can be quantitated using p66 ShcS[36P]-specific antibodies analogously to assays of PY-Shc described in paragraph 1.1.

### Binding Assays

Another embodiment of the instant method identifies anti-proliferative agents based on their ability to interfere with the binding of the Shc A proteins or PY-Shc A proteins or p66 ShcS[36P] proteins or their component domains or polypeptide regions or synthetic analogs thereof, directly or indirectly with receptor PTKs, non-receptor PTKs, PTPases, or downstream cellular effector proteins, each named in the Background section of this application, and which include but are not limited to PTP-PEST, SHIP-1, SHIP-2, Cbl, SHP2, Grb2, EPS8, PLC, polyoma middle T antigen, adaptins, F-actin, focal adhesion kinase, integrins, CEA-CAM, E-cadherin, Gab2, phosphytidyl inosital 3′ kinase, PTEN, PP2A, LDL protein-1, amyloid precursor protein, SOS and others.

(d) Such assays performed in vitro, outside of the cell, using recombinant or synthetic proteins or fragments thereof, in which one component (for example, a PY-peptide that interacts with the Shc SH2 domain, or another PY-peptide that interacts with Shc PTE domain) is immobilized on a solid matrix (for example, physically adsorbed to an ELISA plate or covalently linked to a Sepharose bead or magnetic particle), block any remaining non-specific adsorptive sites using a protein solution such as 1% bovine serum albumin in PBS, and then add a mixture of the agent with the Shc rPTB or rSH2 domain. After allowing this to react for an appropriate time approximating 1 hr, unbound PTB or SH2 domain is washed away and bound PTB or SH2 domain is quantitated by either: i) incorporating a tag such as FLAG into the rSH2 or rPTB domain and detecting with anti-FLAG and appropriate readout systems analogous to those described in paragraph (a); ii) intrinsically or extrinsically label the rPTB or rSH2 domains with a radiolabel or other detectable tag such as biotin; iii) reacting with antibodies specific for the Shc SH2 domain or Shc PTB domain and then quantitating the bound antibodies analogously to paragraph (a). The PTB and SH2 domains are used only as examples: analogous assays can be designed for each of the interacting regions of Shc or the entire Shc molecule, with each of the cellular proteins (or regions or domains or synthetic analogs thereof) that interact with p46, p52 or p66 Shc.

(e) Such assays performed inside living cells, using recombinant or synthetic proteins or fragments thereof, with the cells manipulated or genetically engineered, or not, to have one or more active PTKs or PTPases, and with the cells manipulated or genetically engineered to contain a reporter system capable of sensing binding of the components being tested.

(e) (i) Such assays as (e) in which the cells contain FRET compatible moieties attached to, for example, an rSH2 of Shc and an activated PTK carrying the autophosphorylated tyrosine docking site recognized by the Shc rSH2.

(e) (ii) Such assays as (e) in which yeast “two-hybrid” cells have been genetically engineered to contain both the rSH2 as “bait” and a constitutively activated PTK carrying the autophosphorylated tyrosine docking site recognized by the Shc rSH2 as the “prey” in which effective disrupters of the Shc rSH2 and PY-PTK complex would inhibit yeast growth.

### Therapeutics

Another embodiment of this invention relates to the use of agents that alter the functions or cellular amounts of any of the Shc A isoforms, or agent chemical derivatives thereof or synthetic analogs thereof for the treatment of patients having breast cancer, prostate cancer, other cancers as listed above in the Embodiments.

Such treatment wherein the agents to be administered may be modified for appropriate pharmaceutic properties and combined with delivery-facilitating agents. For example, agents that have primary intracellular targets may need to be chemically modified to render them lipophilic so as to pass through the cell membrane, or they may be incorporated into liposomes to facilitate agent transport across the cell membrane.

### REFERENCES

1. Pawson, T., et al. 1994. Signal transduction and growth control in normal and cancer cells (editorial). Curr Opin Genet Dev 4:1-4.
2. Cohen, G. B., et al. 1995. Modular binding domains in signal transduction proteins. Cell 80:2037-48.
3. Pawson, T. 1995. Protein modules and signalling networks. Nature 373:573-80.
4. Songyang, Z., et al. 1993. SH2 domains recognize specific phosphopeptide sequences. Cell 72:767-78.
5. Xie, Y., et al. 1995. Tyrosine phosphorylation of Shc proteins and formation of Shc/Grb2 complex correlate to the transformation of NIH3T3 cells mediated by the point-mutation activated neu. Oncogene 10:2409-2413.
6. Pelicci, G., et al. 1992. A novel transforming protein (SHC) with an SH2 domain is implicated in mitogenic signal transduction. Cell 70:93-104.
7. Batzer, A. G., et al. 1994. Hierarchy of binding sites for Grb2 and Shc on the epidermal growth factor receptor. Mol Cell Biol 14:5192-201.
8. Buday, L., et al. 1993. Epidermal growth factor regulates p21ras through the formation of a complex of receptor, Grb2 adaptor protein, and Sos nucleotide exchange factor. Cell 73:611-620.
9. Feig, L. A. 1994. Guanine-nucleotide exchange factors: a family of positive regulators of Ras and related GTPases. Curr Opin Cell Biol 6:204-11.
10. Karin, M., et al. 1995. Transcriptional control by protein phosphorylation: signal transmission from the cell surface to the nucleus. Curr Biol 5:747-57.
11. Meyer, S. et al. 1994. Analysis of the role of the Shc and Grb2 proteins in signal transduction by the v-ErbB protein. Mol Cell Biol 14:3253-62.
12. Seger, R., et al. 1995. The MAPK signaling cascade. Faseb J 9:726-35.
13. Segatto, O., et al. 1993. Shc products are substrates of erbB-2 kinase.

Oncogene 8:2105-12.
14. Sutherland, R. L., et al. 1993. Cyclin gene expression and growth control in normal and neoplastic human breast epithelium. J Steroid Biochem Mol Biol 47:99-106.
15. American Cancer Society. 2001. Breast Cancer Facts & Figures 2001-2002. Surveillance Research. In. American Cancer Society, Surveillance Research, 2001.
16. Yarden, Y. 2001. Biology of HER2 and its importance in breast cancer.

Oncology 61 Suppl 2:1-13.
17. Bundred, N. J., et al. 2001. Studies of epidermal growth factor receptor inhibition in breast cancer. Endocr Relat Cancer 8:183-9.
18. Andrechek, E. R., et al. 2000. Tyrosine kinase signalling in breast cancer: tyrosine kinase-mediated signal transduction in transgenic mouse models of human breast cancer. Breast Cancer Res 2:211-6.
19. Dickson, C., et al. 2000. Tyrosine kinase signalling in breast cancer: fibroblast growth factors and their receptors. Breast Cancer Res 2:191-6.
20. Stern, D. F. 2000. Tyrosine kinase signalling in breast cancer: ErbB family receptor tyrosine kinases. Breast Cancer Res 2:176-83.
21. Zhang, X., et al. 2000. Tyrosine kinase signalling in breast cancer: insulin-like growth factors and their receptors in breast cancer. Breast Cancer Res 2:170-5.
22. Hynes, N. E. 2000. Tyrosine kinase signaling in breast cancer. Breast Cancer Res 2:154-7.
23. Dickson, R. B., et al. 1995. Growth factors in breast cancer. Endor Rev 16:559-89.
24. Tuck, A. B., et al. 1996. Coexpression of hepatocyte growth factor and receptor (Met) in human breast carcinoma. Am J Pathol 148:225-32.
25. Nolan, M. K., et al. 1997. Differential roles of IRS-1 and SHC signaling pathways in breast cancer cells. Int J Cancer 72:826-34.
26. Mitchell, P. J., et al. 1994. Cloning and characterisation of cDNAs encoding a novel non-receptor tyrosine kinase, brk, expressed in human breast tumours. Oncogene 9:2383-90.
27. Luttrell, D. K., et al. 1994. Involvement of pp60c-src with two major signaling pathways in human breast cancer. Proc Natl Acad Sci USA 91:83-7.
28. Faisal, A., et al. 2002. Serine/threonine phosphorylation of ShcA. Regulation of protein-tyrosine phosphatase-pest binding and involvement in insulin signaling. J Biol Chem 277:30144-52.
29. Ravichandran, K. S. 2001. Signaling via Shc family adapter proteins. Oncogene 20:6322-30.
30. Filardo, E. J., et al. 2000. Estrogen-induced activation of Erk-1 and Erk-2 requires the G protein-coupled receptor homolog, GPR30, and occurs via trans-activation of the epidermal growth factor receptor through release of HB-EGF. Mol Endocrinol 14:1649-60.
31. Luttrell, L. M., et al. 1997. G-protein-coupled receptors and their regulation: activation of the MAP kinase signaling pathway by G-protein-coupled receptors. Adv Second Messenger Phosphroprotein Res 31:263-77.
32. Buday, L., et al. 1995. Downregulation of the Ras activation pathway by MAP kinase phosphorylation of Sos. Oncogene 11:1327-31.
33. Dong, C., et al. 1996. SOS phosphorylation and disassociation of the Grb2-SOS complex by the ERK and JNK signaling pathways. J Biol Chem 271:6328-32.
34. Hashimoto, A., et al. 1999. Shc regulates epidermal growth factor-induced activation of the JNK signaling pathway. J Biol Chem 274:20139-43.
35. Gotoh, N., et al. 1996. A novel pathway from phosphorylation of tyrosine residues 239/240 of Shc, contributing to suppress apoptosis by IL-3. Embo J 15:6197-204.
36. Gotoh, N., et al. 1997. Tyrosine phosphorylation sites at amino acids 239 and 240 of Shc are involved in epidermal growth factor-induced mitogenic signaling that is distinct from Ras/mitogen-activated protein kinase activation. Mol Cell Biol 17:1824-31.
37. Panchamoorthy, G., et al. 1996. p120cbl is a major substrate of tyrosine phosphorylation upon B cell antigen receptor stimulation and interacts in vivo with Fyn and Syk tyrosine kinases, Grb2 and Shc adaptors, and the p85 subunit of phosphatidylinositol 3-kinase. J Biol Chem 271:3187-94.
38. Baumann, C. A., et al. 2000. CAP defines a second signalling pathway required for insulin-stimulated glucose transport. Nature 407:202-7.
39. Matoskova, B., et al. 1995. Constitutive phosphorylation of eps8 in tumor cell lines: relevance to malignant transformation. Mol Cell Biol 15:3805-12.
40. Scita, G., et al. 1999. EPS8 and E3B1 transduce signals from Ras to Rac Nature 401:290-3.
41. Gu, H., et al. 2000. New role for Shc in activation of the phosphatidylinositol 3-kinase/Akt pathway. Mol Cell Biol 20:7109-20.
42. Nguyen, D. H., et al. 2000. Urokinase-type plasminogen activator stimulates the Ras/Extracellular signal-regulated kinase (ERK) signaling pathway and MCF-7 cell migration by a mechanism that requires focal adhesion kinase, Src, and Shc. Rapid dissociation of GRB2/Sps-Shc complex is associated with the transient phosphorylation of ERK in urokinase-treated cells. J Biol Chem 275:19382-8.
43. Gu, J., et al. 1999. Shc and FAK differentially regulate cell motility and directionality modulated by PTEN. J Cell Biol 146:389-403.
44. Gotoh, N., et al. 1995. The SH2 domain of the Shc suppresses EGF-induced mitogenesis in a dominant negative manner. Oncogene 11:2525-2533.
45. Pelicci, G., et al., 1995. The motogenic and mitogenic responses to HGF are amplified by the Shc adaptor protein. Oncogene 10:1631-8.
46. Sasaoka, T., et al. 1994. Evidence for a functional role of Shc proteins in mitogenic signaling induced by insulin, insulin-like growth factor-1, and epidermal growth factor. J. Biol Chem 269:13689-94.
47. Rozakis-Adcock, M., et al. 1992. Association of the Shc and Grb2/Sem5 SH2-containing proteins is implicated in activation of the Ras pathway by tyrosine kinases. Nature 360:689-92.
48. Ventura, A., et al. 2002. The p66Shc longevity gene is silenced through epigenetic modifications of an alternative promoter. J Biol Chem
49. Migliaccio, E., et al. 1997. Opposite effects of the p52shc/p46shc and p66shc splicing isoforms on the EGF receptor-MAP kinase-fos signaling pathway. Embo J 16:706-16.
50. Okada, S., et al. 1997. The 66-kDa Shc isoform is a negative regulator of the epidermal growth factor-stimulated mitogen-activated protein kinase pathway. J Biol Chem 272:28042-9.
51. Migliaccio, E., et al., 1999. The p66shc adaptor protein controls oxidative stress response and life span in mammals. Nature 402:309-13.
52. Nemoto, S., et al. 2002. Redox regulation of forkhead proteins through a p66shc-dependent signaling pathway. Science 295:2450-2.
53. Brown, N. S., et al. 2001. Hypoxia and oxidative stress in breast cancer. Oxidative stress: its effects on the growth, metastatic potential and response to therapy of breast cancer. Breast Cancer Res 3:323-7.
54. Irani, K., et al. 1997. Mitogenic signaling mediated by oxidants in Ras-transformed fibroblasts (see comments). Science 275:1649-52.
55. Dankort, D., et al. 2001. Multiple ErbB-2/Neu Phosphorylation Sites Mediate Transformation through Distinct Effector Proteins. J Biol Chem 276:38921-B.
56. Webster, M. A., et al. 1998. Requirement for both Shc and phosphatidylinositol 3′ kinase signaling pathways in polyomavirus middle T-mediating mammary tumorigenesis. Mol Cell Biol 18:2344-59.
57. Stevenson, L. A., et al. 1998. Constitutively tyrosine phosphorylated p52 Shc in breast cancer cells: Correlation with ErbB2 and p66 Shc expression. Breast Cancer Research & Treatment 49:119-128.
58. Clark, J. W., et al. 1996. Effects of tyrosine kinase inhibitors on the proliferation of human breast cancer cell lines and proteins important in the Ras signaling pathway. Int J Cancer 65:186-91.
59. Stevenson, L. E. 1998. The role of Shc adapter proteins in breast cancer. In Pathobiology, p. 163. Brown University, Providence.
60. Stevenson, L. A., et al. 1999. Shc dominant negative disrupts cell cycle progression in both G0/G1 and G2/M of ErbB2 positive breast cancer cells. Cell Growth & Differentiation 10:61-71.
61. Davol, P. A. et al. 2001. Requirement for Shc Phosphorylation in Insulin-Like Growth Factor-1 Signaling to Mitogen-Activated Protein Kinase Through the Epidermal Growth Factor Receptor in the Prostate Adenocarcinoma Cell Line PC-3. Proceedings AACR, Vol. 42 42:5156.
62. Collins, L. R., et al. 1999. Bifurcation of Cell migratory and proliferative signaling by the adaptor protein Shc. J Cell Biol 147:1561-8.
63. Trusolino, L., et al. 2001. A signaling adapter function for alpha6beta4 integrin in the control of HGF-dependent invasive growth. Cell 107:643-54.
64. Besson, A., et al. 1999. PTEN/MMAC1/TEP1 in signal transduction and tumorigensis. Eur J Biochem 263:605-11.
65. Hamilton, J. A., et al. 2000. The expression profile for the tumour suppressor gene PTEN and associated polymorphic markers. Br J Cancer 82:1671-6.
66. Hayes, D. F. et al. 2001. Prognostic factors in breast cancer: current and new predictors of metastasis. J Mammary Gland Biol Neoplasia 6:375-92.
67. Gresham, J., et al. 1998. Involvement of Shc in the signaling response of human prostate tumor cell lines to epidermal growth factor. Int J Cancer 77:923-7.
68. Pietrzkowski, Z. et al. 1993. Inhibition of growth of prostatic cancer cell lines by peptide analogues of insulin-like growth factor 1. Cancer Res 53:1102-6.
69. Boney, C. M., et al. 2000. The critical role of Shc in insulin-like growth factor-I-mediated mitogenesis and differentiation in 3T3-L1 preadipocytes. Mol Endocrinol 14:805-13.
70. Boylan, J. M., et al. 1998. Uncoupling of hepatic, epidermal growth factor-mediated mitogen-activated protein kinase activation in the fetal rat. J Biol Chem 273:3784-90.
71. Zhao, H., et al. 1998. Insulin receptor-mediated dissociation of Grb2 from Sos involves phosphorylation of Sos by kinase(s) other than extracellular signal-regulated kinase. J Biol Chem 273:12061-7.
72. Singh, A. et al. 1991. Insulin-like growth factor type I and insulin-like growth factor type II stimulate oestradiol-17 beta hydroxysteroid dehydrogenase (reductive) activity in breast cancer cells. J Endocrinol 129:R5-8.

1. A method for treating a subject afflicted with a proliferative disorder comprising administering to the subject a therapeutically effective amount of an agent which inhibits the expression or activity of p46 Shc and/or p52 Shc in the subject.

2. (canceled)

3. A method for treating a subject afflicted with a proliferative disorder comprising administering to the subject a therapeutically effective amount of an agent which increases the level of phosphorylated p66 Shc in the subject.

4. A method for inhibiting the expression or activity of p46 Shc and/or p52 Shc in a cell comprising delivering to the cell an agent that inhibits the expression or activity of p46 Shc and/or p52 Shc in the cell.

5. (canceled)

6. A method for increasing the level of phosphorylated p66 Shc in a cell comprising delivering to the cell an agent which increases the level of phosphorylated p66 Shc in the cell.

7. The method of claim 1, wherein the agent is selected from the group consisting siRNA, a ribozyme, or a DNAzyme.

8. The method of claim 3, wherein the agent specifically inhibits the dephosphorylation of the Ser36 residue of phosphorylated p66 Shc in the subject.

9. The method of claim 3, wherein the agent is a p66 Shc-encoding expression vector.

10. The method of claim 1, wherein the subject is human.

11. The method of claim 1, wherein the proliferative disease is prostate cancer, ovarian cancer or breast cancer.

12. The method of claim 4, wherein the cell is a prostate cancer cell, an ovarian cancer cell or a breast cancer cell.

13. A method for determining whether an agent inhibits the phosphorylation of p46 Shc or p52 Shc comprising:
(a) contacting p46 Shc or p52 Shc with the agent under conditions which, in the absence of the agent, would permit phosphorylation thereof;
(b) determining the degree to which the p46 Shc or p52 Shc has been phosphorylated; and
(c) comparing the degree of phosphorylation measured in step (b) with the degree of phosphorylation measured in the absence of the agent, a greater degree of phosphorylation in the absence of the agent indicating that the agent inhibits phosphorylation of the p46 or p52 Shc.

14. A method for determining whether an agent inhibits the dephosphorylation of p66 Shc comprising:
(a) contacting phosphorylated p66 Shc with the agent under conditions which, in the absence of the agent, would permit dephosphorylation thereof;
(b) determining the degree to which the p66 Shc has been dephosphorylated; and
(c) comparing the degree of dephosphorylation measured in step (b) with the degree of dephosphorylation measured in the absence of the agent, a greater degree of dephosphorylation in the absence of the agent indicating that the agent inhibits dephosphorylation of the p66 Shc.

15. A method for determining whether an agent inhibits the binding of a Shc A protein with a protein to which the Shc A protein must bind in a cell in order to carry out its proliferative function comprising:
(a) contacting (i) the protein to which Shc A binds, or a Shc A-binding portion thereof, with (ii) Shc A or a suitable portion thereof, in the presence of the agent under conditions which, in the absence of the agent, permit binding;
(b) determining the degree of binding; and
(c) comparing the degree of binding measured in step (b) with the degree of bindng measured in the absence of the agent, a greater degree of binding in the absence of the agent indicating that the agent inhibits the binding between the Shc A protein and the protein to which it must bind in a cell.

16. An article of manufacture comprising (a) a packaging material having therein an agent which inhibits the expression of p46 Shc and/or p52 Shc in a subject; and (b) a label indicating a use for the agent in treating a proliferative disorder in a subject.

17. An article of manufacture comprising (a) a packaging material having therein an agent which inhibits the activity of p46 Shc and/or p52 Shc in a subject; and (b) a label indicating a use for the agent in treating a proliferative disorder in a subject.

18. An article of manufacture comprsing (a) a packaging material having therein an agent which increases the level of phosphorylated p66 Shc in a subject; and (b) a label indicating a use for the agent in treating a proliferative disorder in a subject.

19. The method of claim 4, wherein the agent is selected from the group consisting siRNA, a ribozyme, or a DNAzyme.

20. The method of claim 6, wherein the agent specifically inhibits the dephosphorylation of the Ser36 residue of phosphorylated p66 Shc in the subject.

21. The method of claim 6, wherein the agent is a p66 Shc-encoding expression vector.

22. The method of claim 3, wherein the subject is human.

23. The method of claim of claim 3, wherein the proliferative disease is prostate cancer, ovarian cancer or breast cancer.

24. The method of claim 6, wherein the cell is a prostate cancer cell, an ovarian cancer cell or a breast cancer cell.
